(19) United States
(12) Patent Application Publication
(10) Pub. No.: US 2023/0045685 A1
(43) Pub. Date: Feb. 9, 2023

(54) COPPER CLUSTERS, COMPOSITION, AND METHOD FOR TREATMENT OF LIVER CIRRHOSIS

(71) Applicant: Wuhan Vast Conduct Science Foundation CO., LTD., WUHAN (CN)

(72) Inventor: Taolei SUN, WUHAN (CN)

(73) Assignee: Wuhan Vast Conduct Science Foundation CO., LTD., WUHAN (CN)

(21) Appl. No.: 17/758,309

(22) PCT Filed: Nov. 20, 2020
(86) PCT No.: PCT/CN2020/130028

§ 371 Date: Jul. 1, 2022

(30) Foreign Application Priority Data
Mar. 16, 2020 (CN) PCT/CN2020/079505

(51) Int. Cl. A61K 33/34; A61K 47/36; A61K 47/62

(52) U.S. Cl. CPC A61K 33/34; A61K 47/36; A61K 47/62

(57) ABSTRACT

Use of ligand-bound copper clusters (CuCs) and composition comprising the ligand-bound CuCs to treat liver cirrhosis in a subject. Use of ligand-bound copper clusters (CuCs) to manufacture a medication for the treatment of liver cirrhosis in a subject.

### CROSS-REFERENCE TO RELATED APPLICATIONS

This application is a National Phase Entry of International Application No. PCT/CN2020/130028, filed 19 Nov. 2020, which claims benefit of International Application No. PCT/CN2020/079505, filed 16 Mar. 2020; all of these applications are incorporated herein by reference.

### FIELD OF THE INVENTION

The present invention relates to ligand-bound copper clusters (CuCs), compositions comprising ligand-bound CuCs, and their use for treatment of liver cirrhosis. The present invention further relates to method of using ligand-bound CuCs and compositions for treatment of liver cirrhosis.

### BACKGROUND OF THE INVENTION

The liver is the largest solid organ in a human body, and performs many important functions including: making blood proteins that aid in clotting, transporting oxygen, and helping the immune system; storing excess nutrients and returning some of the nutrients to the bloodstream; manufacturing bile to help digest food; helping the body store sugar (glucose) in the form of glycogen; ridding the body of harmful substances in the bloodstream, including drugs and alcohol; and breaking down saturated fat and producing cholesterol.

Liver cirrhosis is a slowly progressive disease, being developed over many years due to long-term, continuous damage to the liver. Along with the development of liver cirrhosis, healthy liver tissue is gradually destroyed and replaced by scar tissue. The scar tissue blocks the flow of blood through the liver and slows the liver's ability to process nutrients, hormones, drugs, and natural toxins. It also reduces the production of proteins and other substances made by the liver. Cirrhosis may eventually lead to liver failure that may require a liver transplant and/or liver cancer.

In the early stage of liver cirrhosis, there are no obvious symptoms due to strong liver compensatory function. In its later stage, the symptoms include liver function damage, portal hypertension, upper gastrointestinal bleeding, hepatic encephalopathy, secondary infection, spleen hyperfunction, ascites, canceration and other complications. Liver cirrhosis results from gradual liver deformation and hardening. Histopathologically, liver cirrhosis is characterized by extensive hepatic cell necrosis, nodular regeneration of residual hepatocytes, connective tissue hyperplasia and fibrous septum formation, leading to the destruction of hepatic lobular structure and the formation of pseudolobules.

Liver cirrhosis has different causes. Some people with cirrhosis have more than one cause of liver damage. The common causes of cirrhosis include long-term alcohol abuse, chronic hepatitis B and C infection, fatty liver disease, toxic metals, genetic diseases, nutrition disorders, industrial poisons, drugs, circulation disorders, metabolic disorders, cholestasis, schistosomiasis, etc.

Liver cirrhosis could be diagnosed by many tests/techniques. For example, blood test could suggest liver cirrhosis if the levels of the liver enzymes including alanine transaminase (ALT), aspartate transaminase (AST) and alkaline phosphatase (ALP), and bilirubin are increased and the levels of blood proteins are decreased.

Currently, while treatments can delay the progress of liver cirrhosis by dealing with its causes, there is no specific treatments for liver cirrhosis.

### SUMMARY OF THE INVENTION

The present invention provides the use a ligand-bound copper cluster (CuC) to treat a subject with liver cirrhosis, wherein said ligand-bound CuC comprises a copper core; and a ligand, wherein a ligand, wherein the ligand binds to the copper core, forming the ligand-bound copper cluster (CuC).

In certain embodiments of the use for treatment, the copper core has a diameter in the range of 0.5-5 nm. In certain embodiments, the copper core has a diameter in the range of 0.5-3 nm.

In certain embodiments of the use for treatment, the ligand is one selected from the group consisting of thymine, thymine-modified hyaluronic acid (TMHA), L-cysteine and its derivatives, D-cysteine and its derivatives, cysteine-containing oligopeptides and their derivatives, and other thiol-containing compounds.

In certain embodiments of the use for treatment, the L-cysteine and its derivatives are selected from the group consisting of L-cysteine, N-isobutyryl-L-cysteine (L-NIBC), and N-acetyl-L-cysteine (L-NAC), and the D-cysteine and its derivatives are selected from the group consisting of D-cysteine, N-isobutyryl-D-cysteine (D-NIBC), and N-acetyl-D-cysteine (D-NAC).

In certain embodiments of the use for treatment, the cysteine-containing oligopeptides and their derivatives are cysteine-containing dipeptides, cysteine-containing tripeptides, or cysteine-containing tetrapeptides.

In certain embodiments of the use for treatment, the cysteine-containing dipeptides are selected from the group consisting of L(D)-cysteine-L(D)-arginine dipeptide (CR), L(D)-arginine-(D)L-cysteine dipeptide (RC), L(D)-histidine-L(D)-cysteine dipeptide (HC), and L(D)-cysteine-L(D)-histidine dipeptide (CH).

In certain embodiments of the use for treatment, the cysteine-containing tripeptides are selected from the group consisting of glycine-L(D)-cysteine-L(D)-arginine tripeptide (GCR), L(D)-proline-L(D)-cysteine-L(D)-arginine tripeptide (PCR), L(D)-lysine-L(D)-cysteine-L(D)-proline tripeptide (KCP), and L(D)-glutathione (GSH).

In certain embodiments of the use for treatment, the cysteine-containing tetrapeptides are selected from the group consisting of glycine-L(D)-serine-L(D)-cysteine-L(D)-arginine tetrapeptide (GSCR), and glycine-L(D)-cysteine-L(D)-serine-L(D)-arginine tetrapeptide (GCSR).

In certain embodiments of the use for treatment, the other thiol-containing compounds are selected from the group consisting of 1-[(2S)-2-methyl-3-thiol-1-oxopropyl]-L(D)-proline, thioglycollic acid, mercaptoethanol, thiophenol, D-3-trolovol, N-(2-mercaptopropionyl)-glycine, and dodecyl mercaptan.

The present invention also provides the use of a ligand-bound copper cluster (CuC) for manufacture of a medicament for the treatment of liver cirrhosis in a subject, wherein said ligand-bound CuC comprises a copper core; and a ligand, wherein the ligand binds to the copper core, forming the ligand-bound copper cluster (CuC).

In certain embodiments of the use for manufacture, the copper core has a diameter in the range of 0.5-5 nm. In certain embodiments, the copper core has a diameter in the range of 0.5-3 nm.

In certain embodiments of the use for manufacture, the ligand is one selected from the group consisting of thymine, thymine-modified hyaluronic acid (TMHA), L-cysteine and its derivatives, D-cysteine and its derivatives, cysteine-containing oligopeptides and their derivatives, and other thiol-containing compounds.

In certain embodiments of the use for manufacture, the L-cysteine and its derivatives are selected from the group consisting of L-cysteine, N-isobutyryl-L-cysteine (L-NIBC), and N-acetyl-L-cysteine (L-NAC), and the D-cysteine and its derivatives are selected from the group consisting of D-cysteine, N-isobutyryl-D-cysteine (D-NIBC), and N-acetyl-D-cysteine (D-NAC).

In certain embodiments of the use for manufacture, the cysteine-containing oligopeptides and their derivatives are cysteine-containing dipeptides, cysteine-containing tripeptides, or cysteine-containing tetrapeptides.

In certain embodiments of the use for manufacture, the cysteine-containing dipeptides are selected from the group consisting of L(D)-cysteine-L(D)-arginine dipeptide (CR), L(D)-arginine-L(D)-cysteine dipeptide (RC), L(D)-histidine-L(D)-cysteine dipeptide (HC), and L(D)-cysteine-L(D)-histidine dipeptide (CH).

In certain embodiments of the use for manufacture, the cysteine-containing tripeptides are selected from the group consisting of glycine-L(D)-cysteine-L(D)-arginine tripeptide (GCR), L(D)-proline-L(D)-cysteine-L(D)-arginine tripeptide (PCR), L(D)-lysine-L(D)-cysteine-L(D)-proline tripeptide (KCP), and L(D)-glutathione (GSH).

In certain embodiments of the use for manufacture, the cysteine-containing tetrapeptides are selected from the group consisting of glycine-L(D)-serine-L(D)-cysteine-L(D)-arginine tetrapeptide (GSCR), and glycine-L(D)-cysteine-L(D)-serine-L(D)-arginine tetrapeptide (GCSR).

In certain embodiments of the use for manufacture, the other thiol-containing compounds are selected from the group consisting of 1-[(2S)-2-methyl-3-thiol-1-oxopropyl]-L(D)-proline, thioglycollic acid, mercaptoethanol, thiophenol, D-3-trolovol, N-(2-mercaptopropionyl)-glycine, and dodecyl mercaptan.

The objectives and advantages of the invention will become apparent from the following detailed description of preferred embodiments thereof in connection with the accompanying drawings.

### DESCRIPTION OF THE DRAWINGS

Preferred embodiments according to the present invention will now be described with reference to the Figures, in which like reference numerals denote like elements.

FIG. 1 shows characterization data of L-GSH-CuCs. (A) A typical transmission electron microscopic (TEM) image of GSH-CuCs. (B) Size distribution of GSH-CuCs calculated from TEM images. (C) X-ray photoelectron spectroscopy (XPS) spectrum of 2p_{3/2} and 2p_{1/2} electrons of Cu(0) in GSH-CuCs. (D) Comparison between Fourier transform infrared (FT-IR) spectroscopies of GSH-CuCs (upper) and GSH (lower). (E) Fluorescent excitation (left) and emission spectra (right) of GSH-CuCs.

FIG. 2 presents bar graphs showing the effects of different doses of Cu-1 and Cu-2 on serum (A) ALT, (B) AST, (C) TBIL, (D) MAO and (E) ALB levels in cirrhotic model mice, where 1) denotes the blank control group, 2) the model group, 3) the positive group treated with sorafenib, 4) Cu-1 low dose group, 5) Cu-1 high dose group, 6) Cu-2 low dose group, and 7) Cu-2 high dose group.

FIG. 3 presents HE staining images: (A) the blank control group; (B) the model group; (C) the positive control group; (D) Cu-1 low dose group; (E) Cu-1 high dose group.

### DETAILED DESCRIPTION OF THE EMBODIMENTS

The present invention may be understood more readily by reference to the following detailed description of certain embodiments of the invention.

Throughout this application, where publications are referenced, the disclosures of these publications are hereby incorporated by reference, in their entireties, into this application in order to more fully describe the state of art to which this invention pertains.

Ligand-bound copper clusters are composed of copper cores formed by two to several hundreds of copper atoms, and ligands. The ligands as part of the ligand-bound copper cluster molecules bind to the copper cores, forming the ligand-bound copper clusters being stable in solution. Because of the low contrast of copper atoms, it is difficult to give a very accurate size of copper cores by TEM. It is commonly accepted that the sizes of copper cores in ligand-bound copper clusters are in the range of 0.5-5 nm by TEM.

The present invention provides ligand-bound copper clusters (CuCs), where one or more ligands bind to a copper core. The binding of ligands with copper cores means that ligands form stable-in-solution complexes with copper cores through covalent bond, hydrogen bond, electrostatic force, hydrophobic force, van der Waals force, etc In certain embodiments, the copper core has a diameter in the range of 0.5-5 nm, preferably in the range of 0.5-3 nm, and more preferably in the range of 0.5-2.5 nm.

In certain embodiments, the ligands include, but not limited to, thymine, thymine-modified hyaluronic acid (TMHA), L-cysteine, D-cysteine and other cysteine derivatives such as N-isobutyryl-L-cysteine (L-NIBC), N-isobutyryl-D-cysteine (D-NIBC), N-acetyl-L-cysteine and N-acetyl-D-cysteine, cysteine-containing oligopeptides and their derivatives including, but not limited to, dipeptides, tripeptide, tetrapeptide and other peptides containing cysteine, such as L(D)-cysteine-L(D)-arginine dipeptide (CR), L(D)-arginine-L(D)-cysteine dipeptide (RC), L(D)-cysteine L(D)-histidine (CH), glycine-L(D)-cysteine-L(D)-arginine tripeptide (GCR), L(D)-proline-L(D)-cysteine-L(D)-arginine tripeptide (PCR), L(D)-glutathione (GSH), glycine-L(D)-serine-L(D)-cysteine-L(D)-arginine tetrapeptide (GSCR) and glycine-L(D)-cysteine-L(D)-serine-L(D)-arginine tetrapeptide (GCSR), and other thiol-containing compounds, such as one or more of 1-[(2S)-2-methyl-3-thiol-1-oxopropyl]-L(D)-proline, thioglycollic acid, mercaptoethanol, thiophenol, D-3-trolovol and dodecyl mercaptan.

Ligand-bound CuCs with different ligands can be prepared by methods adopted from literatures (Deng 2018, Jia 2013; Wang 2013).

The present invention provides a composition for treating a subject with liver cirrhosis. In certain embodiments, the composition comprises ligand-bound copper clusters (CuCs), and a pharmaceutically acceptable excipient. In certain embodiments, the excipient is phosphate-buffered solution, or physiological saline. In certain embodiments, the subject is human. In certain embodiments, the subject is a pet animal such as a dog.

The present invention provides a use of the above disclosed ligand-bound CuCs for manufacturing a medication for the treatment of liver cirrhosis in a subject.

The present invention provides a use of the above disclosed ligand-bound CuCs for treating liver cirrhosis in a subject or a method for treating liver cirrhosis in subject using the above disclosed ligand-bound CuCs. In certain embodiments, the method for treatment comprises administering a pharmaceutically effective amount of ligand-bound CuCs to the subject. The pharmaceutically effective amount can be ascertained by routine in vivo studies.

The following examples are provided for the sole purpose of illustrating the principles of the present invention; they are by no means intended to limit the scope of the present invention.

### EMBODIMENTS

### Embodiment 1. Synthesis of TMHA-modified CuCs with TMHA

10 mL of TMHA (DS of 10.5%) solution (0.1 mM, pH 7.0) was gradually heated up to 37° C. to dissolve the TMHA. 2 mL of CuSO₄ (20 mM, pH 7.0) solution was added dropwise and allowed to react for another 20 min in dark at 37° C. Under radiation of UV-light (365 nm), a bright orange-red emission was clearly visible, indicating the successful formation of luminescent TMHA-modified CuCs. Finally, the resultant solution was stored in dark at 4° C. for use. The spherical TMHA-modified CuCs are with a copper core that has diameters in a range of 0.5-3 nm, the average diameters of which are 1.64±0.48 nm.

### Embodiment 2. Synthesis and Characterization of Ligand-Bound CuCs with Different Ligands

2.1 Synthesis of L-Glutathione (GSH)-Bound Copper Clusters (L-GSH-CuCs)

Into 50 ml of water was added 500 mg of glutathione (GSH) to form a GSH solution; under slow stirring, 20 ml of 5 mM Cu(NO₃)₂ solution was added into the GSH solution, resulting in a quick formation of a white suspension. The mixture was slowly heated to 50-60° C. and the heating was continued for 20 min, and then added 1 m NaOH solution drop by drop until the solution turns light yellow, clear and transparent. The product was cooled to room temperature, precipitated by adding several times the volume of ethanol, and repeated three times.

2.2 Synthesis of L-Cysteine-Bound Copper Clusters

50 ml of 10 mM CuCl₂ was slowly added drop by drop into the freshly prepared L-cysteine (50 ml, 10 mM) solution under intense agitation. About 30 minutes later, 0.5 ml NaOH (1M) was slowly added drop by drop to the above solution. The reaction continued for 2 hours. The product was centrifuged at 8000 rpm for 20 min, and the supernatant was stored at 4° C. away from light.

2.3 Synthesis of PEG-Bound Copper Clusters

2.5 g of PEG-SH (molecular weight 2000 or 5000) was dissolved in 100 ml of ultrapure water at room temperature, and 4 ml of 0.5 M Cu(NO₃)₂ solution was added drop by drop under intense agitation. The mixture was stirred at room temperature for a period of time until its color faded and milky white color was gradually formed. Then the gel was gradually heated to 80° C. and maintained for 15 minutes. 3 M NaOH solution was added drop by drop until the solution became clear and transparent. The product was centrifuged at 8000 rpm for 20 min, and the final product was lyophilized in a freeze dryer to obtain a solid sample.

2.4 Synthesis of Ligand-Bound Copper Clusters with Other Ligands

Ligand-bound copper clusters with other ligands can also be synthesized by the above method, and the specific synthesis method needs to be slightly modified with some solvents and operations. Other ligands include thymine, L(D)-cysteine and other cysteine derivatives such as N-isobutyryl-L-cysteine (L-NIBC), N-isobutyryl-D-cysteine (D-NIBC), N-acetyl-L-cysteine and N-acetyl-D-cysteine, cysteine-containing oligopeptides and their derivatives including, but not limited to, dipeptides, tripeptide, tetrapeptide and other peptides containing cysteine, such as L(D)-cysteine-L(D)-arginine dipeptide (CR), L(D)-arginine-L(D)-cysteine dipeptide (RC), L(D)-cysteine L(D)-histidine (CH), glycine-L(D)-cysteine-L(D)-arginine tripeptide (GCR), L(D)-proline-L(D)-cysteine-L(D)-arginine tripeptide (PCR), L(D)-glutathione (GSH), glycine-L(D)-serine-L(D)-cysteine-L(D)-arginine tetrapeptide (GSCR) and glycine-L(D)-cysteine-L(D)-serine-L(D)-arginine tetrapeptide (GCSR), and other thiol-containing compounds, such as one or more of 1-[(2S)-2-methyl-3-thiol-1-oxopropyl]-L(D)-proline, thioglycollic acid, mercaptoethanol, thiophenol, D-3-trolovol and dodecyl mercaptane.

2.5 Characterization of Ligand-Bound Copper Clusters

The following characterization data of L-GSH-CuCs are shown as an example.

1) Observation of the Morphology by Transmission Electron Microscope (TEM)

The test powders (GSH-CuCs sample) were dissolved in ultrapure water to 2 mg/L as samples, and then test samples were prepared by hanging drop method. The specific method: 5 μL of the samples were dripped on the copper mesh, volatized naturally till the water drop disappeared, and then observe the morphology of the samples by JEM-2100F STEM/EDS field emission high-resolution TEM.

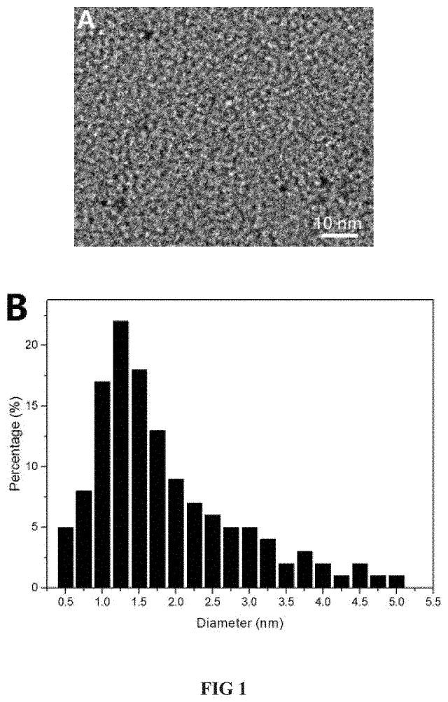

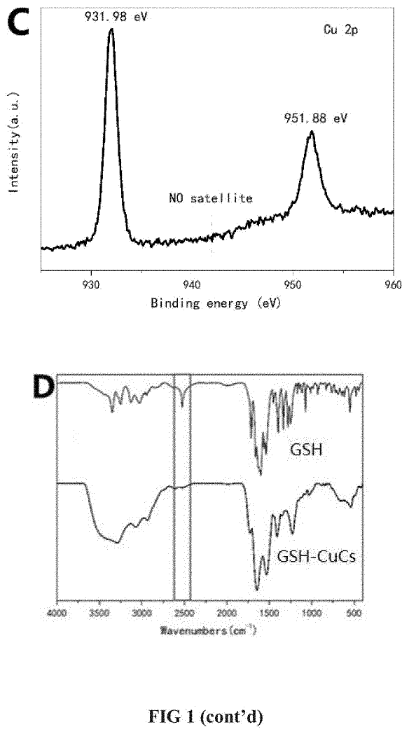

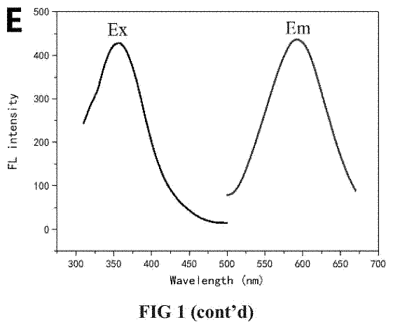

Panel A and panel B of FIG. 1 show a typical SEM image of GSH-CuCs, and their size distribution was calculated from different TEM images. It indicates that GSH-CuCs are well-dispersed and their sizes lie in a range of 0.5-5.0 nm.

2) X-Ray Photoelectron Spectroscopy

The X-ray photoelectron spectroscopy (XPS) spectra was measured on an ESCALAB 250Xi X-ray photoelectron spectrometer. A double-sided conductive adhesive (3 mm×3 mm) was attached to the aluminum foil, the test powder was evenly spread on the double-sided tape and covered with a layer of aluminum foil. The sample was kept under a pressure of 8 MPa for one minute. Remove the residual powder on the surface and then the center sample (1 mm×1 mm) was cut out for XPS testing.

Panel C of FIG. 1 is the XPS spectrum of Cu element in GSH-CuCs. Two peaks appear at 931.98 and 951.88 eV, which can be ascribed to the binding energies of the 2p_{3/2} and 2p_{1/2} electrons of Cu, respectively. The absence of Cu 2p_{3/2} satellite peak around 942.0 eV confirms that the Cu(II) electrons are not present. As the binding energy of Cu(0) is only 0.1 eV away from that of Cu(I), it is not possible to exclude the formation of Cu(I), and the valence state of Cu in the obtained GSH-CuCs most likely lies between 0 and +1.

3) Fourier Transform Infrared (FT-IR) Spectroscopy

The FT-IR spectra was tested on the PerkinElmer LS 55 fluorescence spectrometer. The test powder was dissolved in ultrapure water, and measured at room temperature. The scanning range was 200-800 nm, the sample cell was a standard quartz cuvette with an optical path of 1 cm.

Panel D of FIG. 1 shows a comparison between FT-IR spectroscopies of GSH-CuCs (upper) and GSH (lower). GSH exhibits a number of characteristic IR bands, i.e., COOH⁻ (1,390 and 1,500 cm⁻¹), the N—H stretch (3,410 cm⁻¹), and the N—H bending (1,610 cm⁻¹) of NH₂ group. The peak observed at 2,503 cm⁻¹ can be assigned to the S—H stretching vibrational mode. Corresponding characteristic IR bands can all be found for GSH-CuCs, except for the S—H stretching vibration band (2,503 cm⁻¹). It suggests the cleavage of the S—H bond and the binding of the GSH molecules to the surface of the copper core through the formation of Cu—S bond.

4) Fluorescence Spectroscopy

The test powder was dissolved in ultrapure water and measured by fluorescence spectroscopy at room temperature.

As shown in the panel E of FIG. 1, the GSH-CuCs exhibit red emission with a peak at 595 nm and a corresponding full width at half maximum (FWHM) of approximately 80 nm under the excitation peak at 365 nm. It is worth noting that the FL intensity of the GSH-CuCs will be significant improved when the ethanol was added to the solution due to the aggregation induced emission enhancement. In addition, the large stokes shift (230 nm) indicated good prospects for fluorescent probes and bioimaging.

### Embodiment 3

3.1 Materials and animals

3.1.1 Testing Sample

Cu-01: GSH-modified copper clusters (L-GSH-CuCs), 0.5-5 nm.

Cu-02: Cysteine-modified copper clusters (L-Cys-CuCs), 0.5-5 nm.

All testing samples were prepared following the above described method with slight modification, and their quality was characterized using the above described methods.

3.1.2 Positive Control Sample

Sorafenib.

3.1.3 Animals for Experiments and Groups

70 SPF male C57BL/6N mice, 6-8 weeks old and 16-20 g body weight, were purchased from Beijing Huafukang Experimental Animal Technology Co., Ltd. (production license number: SOCK (Jing) 2019-0008). According to body weight, they were randomly divided into 7 groups (n=10): blank control group, model group, positive control group, Cu-1 low dose group, Cu-1 high dose group, Cu-2 low dose group, Cu-2 high dose group.

3.2 Modeling Protocol

Except for the blank control group, liver cirrhosis model of mice in other groups was prepared by the treatment of carbon tetrachloride (CCl₄)-induction method. The modeling protocoal was as follows: (1) Each mouse was intraperitoneally injected with 10% CCl₄ (diluted with olive oil) at 7 μL/g body weight, twice a week for a total of 8 weeks; mice of the blank control group were injected intraperitoneally with the same amount of olive oil solvent. (2) from the 6th week, two mice were selected and killed 48 hours after the last injection every week. The appearance of the liver was observed. After the appearance was in line with the characteristics of cirrhosis (the 8th week), the liver tissue was fixed with formalin. HE staining and Masson staining were used to evaluate the model of cirrhosis.

3.3 Administration

After the successful modeling, the mice in the positive control group were given intragastrically 25 mg/kg sorafenib; the mice in the low or high dose groups of Cu-1 and Cu-2 were given by intraperitoneal injection at 2.5 or 10 mg/kg respectively of the corresponding test material; and the mice in the blank control group and the model group were given intraperitoneally physiological saline at 10 mL/kg. The administration was once a day for 20 consecutive days.

3.4 Biochemical Testing

After the administration was completed, blood was collected from mouse orbit, and sera were obtained for biochemical testing of albumin (ALbumin, ALB), total bilirubin (TBil), alanine Alanine aminotransferase (ALT), aspartate aminotransferase (AST) and monoamine oxidase (MAO) using Zhongsheng Beikong Kit and biochemical analyzer (Siemens). The detection method was performed in strict accordance with the kit instructions.

Table 1 shows the product information of kits used for biochemical testing

| Serial | | | |
|---|---|---|---|
| number | Kit name | abbreviation | registration number |
| 1 | Albumin Test Kit | ALB | Beijing Food and Drug |
| | (Bromocresol Green Method) | | administration Device (Permit) |
| | | | 2014 No. 2401133 |
| 2 | Total bilirubin test kit (vanadate | TBil | Beijing Food and Drug |
| | oxidation method) | | administration Device (Permit) |
| | | | 2014 No. 2401140 |
| 3 | Alanine aminotransferase test | ALT | Beijing Food and Drug |
| | kit | | administration Device (Permit) |
| | (alanine substrate method) | | 2014 No. 2401158 |
| 4 | Aspartate aminotransferase test | AST | Beijing Food and Drug |
| | kit | | administration Device (Permit) |
| | (aspartic acid substrate method) | | 2014 No. 2401157 |
| 5 | Monoamine oxidase test kit | MAO | Beijing Food and Drug |
| | (glutamic acid dehydrogenase | | administration Device (Permit) |
| | method) | | 20162401129 |

3.5 Pathological Examination

3.5.1 HE Staining

After euthanasia, the mouse liver tissue samples were fixed with 4% paraformaldehyde fixative for more than 48 h. After fixation, the liver samples were dehydrated with alcohol gradient and treated with xylene and ethanol. Then, the liver tissues were then dipped in wax and embedded. After the embedded material being trimmed, attached, and repaired, the liver tissues were sliced with a paraffin microtome, and the slices were with a thickness of 4 μm. The main process of HE staining is as follows: After baked in the oven at 65° C., the slices were treated with xylene and dehydrated with gradient ethanol. The slices were sequentially stained with hematoxylin, blue color-enhancing solution, and 0.5% eosin, then treated with gradient ethanol and xylene and sealed with neutral gum. The fibrosis of liver tissue was observed with a microscope.

3.5.2 Masson Staining

After baked, mouse liver tissue slices were dewaxed and dehydrated. After chromizing, the nucleus was stained with Regaud's hematoxylin staining solution. After washing with water, the slices were stained with Masson's Ponceau Red Acidic Fuchsin, and the slices were dipped in a 2% glacial acetic acid aqueous solution and differentiated with a 1% phosphomolybdic acid solution. After directly stained with aniline blue or light green solution, the slices were dipped in a 0.2% glacial acetic acid aqueous solution for a while, then transparentized with 95% alcohol, anhydrous alcohol and xylene, and then sealed with neutral gum. Liver tissue was observed with a microscope.

3.6 Experimental Results

3.6.1 Successful Modeling

The livers of mice in the model group were divided into round or oval masses of different sizes by proliferating fibrous septa. The serum ALT, TBil, and AST indexes increased significantly compared to that of the blank control group, the serum ALB significantly decreased compared to the blank control group, and the MAO index was no significant difference from the control group, but the value also increased. All the above results suggest that this experimental modeling was successful.

3.6.2 Effects of Test Drugs on Alanine Aminotransferase (ALT), Total Bilirubin (TBil), Aspartate Aminotransferase (AST), Monoamine Oxidase (MAO) and Albumin (ALB).

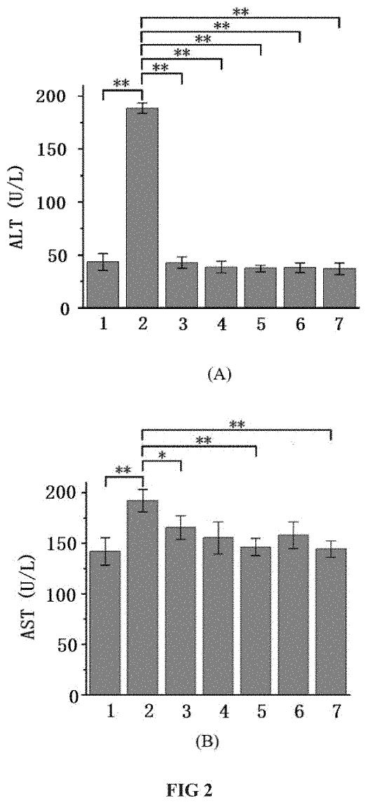

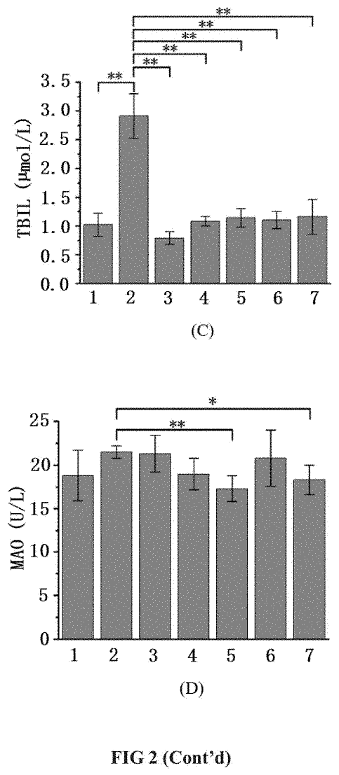

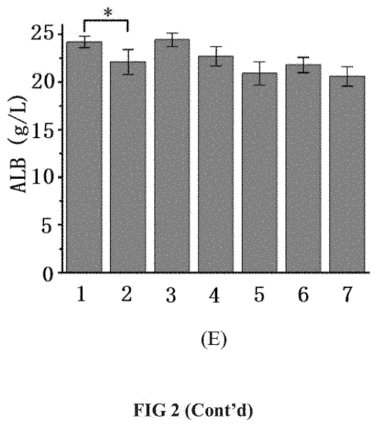

As shown in FIG. 2A, compared with the blank control group, ALT activity of the model group is increased extremely significantly (increased from 43.5±8.1 U/L to 188.5±4.9 U/L; P<0.01), indicating that the liver functions of the model group mice underwent pathological changes. Compared with the model group, the low and high dose of Cu-1 and Cu-2 (lowest is 37.0±5.7 U/L; highest is 38.6±5.6 U/L), as well as the positive control (42.8±5.4 U/L), significantly reduced ALT activity to the level of the blank control group (P<0.01).

As shown in FIG. 2B, compared to the blank control group, AST activity of the model group is increased significantly (increased from 141.8±13.5 U/L to 192.0±11.3 U/L; P<0.05). Administration of high dose Cu-1 and Cu-2 can significantly reduce AST activity to 146.3±8.4 U/L or 144.3±8.1 U/L, respectively; these are in the same level as that of the blank control group (141.8±13.5 U/L), but are significantly lower than that of the model group (P<0.01). The positive control can also reduce AST activity (165.5±11.6 U/L; P<0.05), but the reduction extent is lower than that of high dose groups of Cu-1 and Cu-2.

As shown in FIG. 2C, TBil concentration of the model group is significantly higher than that of the blank control group (increased from 1.02±0.20 μmon to 2.91±0.39 μmon; P<0.01). Compared with the model group, administration of low and high dose Cu-1 and Cu-2 significantly reduced the level of serum TBIL (highest 1.16±0.30 μmon; lowest 1.08±0.08 μmon; P<0.01); these are close to that of the blank control group (1.02±0.20 μmon; P<0.01).

As shown in FIG. 2D, MAO activity of the model group (21.5±0.7 U/L) is higher than that of the blank control group (18.8±2.9 U/L), but there is no statistically significant difference, indicating that the change of MAO activity indicator is not apparent in CCl₄-induced liver cirrhosis in a mouse model. However, compared with the model group, the high dose Cu-1 and Cu-2 significantly reduced the serum MAO activity to 17.3±1.5 U/L (P<0.01) or (18.3±2.1 U/L; P<0.05), and the effect was better than that of the positive control.

As shown in FIG. 2E, ALB level of the model group (24.2±0.6 g/L) is significantly lower than that of the blank control group (22.1±1.3 g/L) (P<0.05), indicating that CCl₄ treatment could significantly decrease the ALB serum level. However, Cu-1 and Cu-2 have no significant effect on serum ALB level.

The above results showed that copper clusters (CuCs) decreased the levels of ALT, AST, TBIL and Mao in a dose-dependent manner, suggesting that the liver function of mice was restored, and its effect is better than that of positive control drugs at least in some indicators.

3.6.3 Pathological Analyses

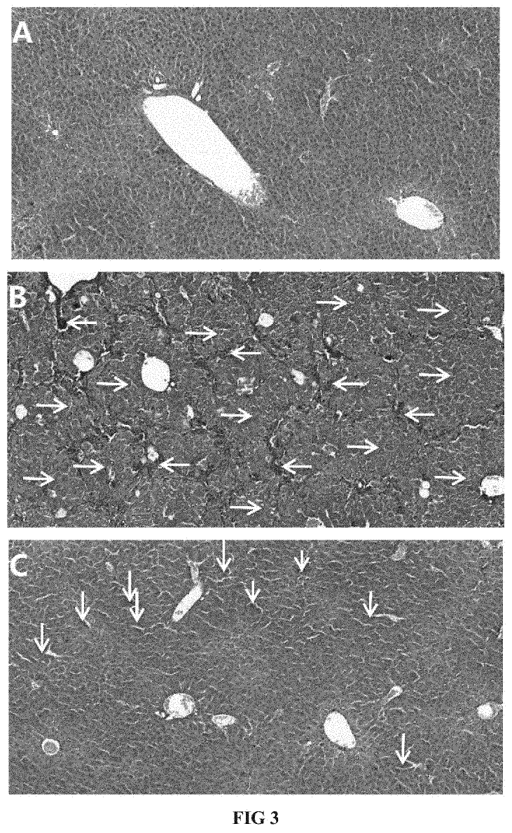

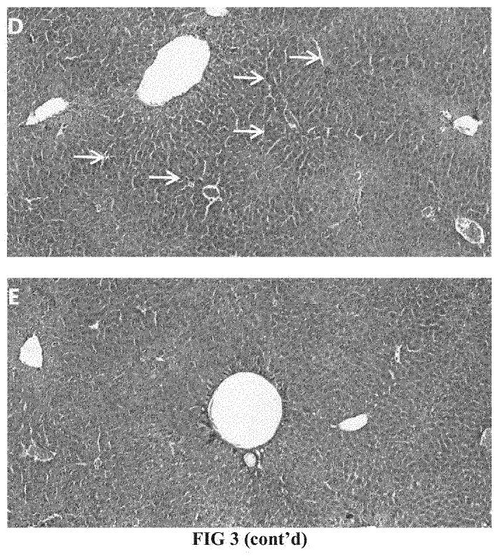

Liver cirrhosis is pathologically characterized by diffuse fibrosis of the liver tissue and formation of pseudolobules. The results of HE staining pathological analyses showed that as presented in FIG. 3A, the normal liver tissue from the mice of the blank control group had clear structure, intact liver lobules, neatly arranged hepatocytes, radial arrangement being centered on the central vein, normal nucleus of hepatocytes, and only a small amount of fibrous tissue in the catchment area. As shown in FIG. 3B, in the liver tissue of the model group, the hepatocytes were disordered, balloon-like structures appeared, the hepatic lobules nearly disappeared, pseudolobules (as pointed to by right-pointed arrows in FIG. 3B) were abundantly formed, and a large number of proliferated protofibrils were present in the liver tissues, forming round- or oval-shaped fibrous septa (as pointed to by left-pointed arrows in FIG. 3B). As shown in FIG. 3C, compared with the model control group, the positive control group showed significant reduction of liver damages; the hepatocytes evidently have neat arrangement; fibrous hyperplasia, while increased, apparently reduced, not forming fibrous septa; pseudolobules nearly disappeared; but compared with normal liver tissues, the liver tissues in the positive control group showed apparent increases of inter-cellular gaps (as pointed to by downward-pointed arrows). Compared with the model control group, the 2 groups administered with copper clusters drugs (Cu-1 and Cu-2) showed that their hepatocytes significantly recovered from liver damages, as evidenced by apparent reduction of fibrous hyperplasia and pseudolobules, and that the recovery is dose-dependent to a certain extent.

FIG. 3D and FIG. 3E show the HE images that showed the effects of the exemplary Cu-1 low and high dose drug administration respectively on the recovery of liver damages. As shown in FIG. 3D, Cu-1 low dose drug administration group showed relatively neat arrangement of hepatocytes, near disappearance of pseudolobules, evident reduction of fibrous hyperplasia, but the inter-hepatocytes gaps, compared with normal liver tissues, are increased to a certain extent (as pointed to by downward-pointed arrows in FIG. 3D). As shown in FIG. 3E, in comparison with Cu-1 low dose drug administration group, Cu-1 high dose drug administration group had even better effects of reduction of liver damages, complete disappearance of pseudolobules, no observation of fibrous hyperplasia, no discernable increases of inter-hepatocytes gaps, and no apparent difference from normal liver tissues. In conclusion, Cu-1 drug showed better effects on recovery of liver damages than the positive control drug.

The results from Masson staining provided the same conclusions as did the results of HE staining.

Cu-2 drug also showed similar effects of Cu-1 drug; no detailed description is needed.

In summary, Cu-1 and Cu-2 test drugs significantly reduced liver fibrosis and liver pseudolobules. The test results of liver function indicators also showed the recovery of liver function. The most significant changes were alanine aminotransferase (ALT) and total bilirubin (TBil). Aspartate aminotransferase (AST) and monoamino oxidase (MAO) also recovered significantly, while albumin (ALB) did not change significantly. The two test substances may significantly improve liver function and the liver pathological structure in cirrhotic mice; furthermore, the total effects of copper clusters are better than that of the positive control Sorafenib. These results provide experimental basis for further application in the future.

Other sized L-Cys-CuCs and L-GSH-CuCs, and other ligand-bound CuCs with different sizes also have the similar effects, while their effects vary to certain extents. They would not be described in detail here.

While the present invention has been described with reference to particular embodiments, it will be understood that the embodiments are illustrative and that the invention scope is not so limited. Alternative embodiments of the present invention will become apparent to those having ordinary skill in the art to which the present invention pertains. Such alternate embodiments are considered to be encompassed within the scope of the present invention. Accordingly, the scope of the present invention is defined by the appended claims and is supported by the foregoing description.

### REFERENCES

Deng H. H. et al. An ammonia-based etchant for attaining copper nanoclusters with green fluorescence emission. Nanoscale, 2018, 10, 6467.
Jia X. et al. Cu Nanoclusters with Aggregation Induced Emission Enhancement. Small, 2013, DOI: 10.1002/smll.201300896.
Wang C. and Huang Y. GREEN ROUTE TO PREPARE BIOCOMPATIBLE AND NEAR INFRARED THIOLATE-PROTECTED COPPER NANOCLUSTERS FOR CELLULAR IMAGING. NANO: Brief Reports and Reviews. 2013, 8(5): 1350054 (10 pages).

1. A method for treating a subject with liver cirrhosis, wherein the method comprises:
administering a composition to the subject with liver cirrhosis;
wherein the composition comprises a ligand-bound copper cluster; and
a pharmaceutically acceptable excipient;
wherein the ligand-bound copper cluster comprises:
a copper core; and
a ligand, wherein the ligand, binds to the copper core, forming the ligand-bound copper cluster.

2. The method of claim 1, wherein the copper core has a diameter in the range of 0.5-5 nm.

3. The method of claim 1, wherein the copper core has a diameter in the range of 0.5-3 nm.

4. The method of claim 1, wherein the ligand is one selected from the group consisting of thymine, thymine-modified hyaluronic acid (TMHA), L-cysteine and its derivatives, D-cysteine and its derivatives, cysteine-containing oligopeptides and their derivatives, and other thiol-containing compounds.

5. The method of claim 4, wherein the L-cysteine and its derivatives are selected from the group consisting of L-cysteine, N-isobutyryl-L-cysteine (L-NIBC), and N-acetyl-L-cysteine (L-NAC), and wherein the D-cysteine and its derivatives are selected from the group consisting of D-cysteine, N-isobutyryl-D-cysteine (D-NIBC), and N-acetyl-D-cysteine (D-NAC).

6. The method of claim 4, wherein the cysteine-containing oligopeptides and their derivatives are cysteine-containing dipeptides, cysteine-containing tripeptides, or cysteine-containing tetrapeptides.

7. The method of claim 6, wherein the cysteine-containing dipeptides are selected from the group consisting of L(D)-cysteine-L(D)-arginine dipeptide (CR), L(D)-arginine-L(D)-cysteine dipeptide (RC), L(D)-histidine-L(D)-cysteine dipeptide (HC), and L(D)-cysteine-L(D)-histidine dipeptide (CH).

8. The method of claim 6, wherein the cysteine-containing tripeptides are selected from the group consisting of glycine-L(D)-cysteine-L(D)-arginine tripeptide (GCR), L(D)-proline-L(D)-cysteine-L(D)-arginine tripeptide (PCR), L(D)-lysine-L(D)-cysteine-L(D)-proline tripeptide (KCl′), and L(D)-glutathione (GSH).

9. The method of claim 6, wherein the cysteine-containing tetrapeptides are selected from the group consisting of glycine-L(D)-serine-L(D)-cysteine-L(D)-arginine tetrapeptide (GSCR), and glycine-L(D)-cysteine-L(D)-serine-L(D)-arginine tetrapeptide (GCSR).

10. The method of claim 4, wherein the other thiol-containing compounds are selected from the group consisting of 1-[(2S)-2-methyl-3-thiol-1-oxopropyl]-L(D)-proline, thioglycollic acid; mercaptoethanol, thiophenol, D-3-trolovol, N-(2-mercaptopropionyl)-glycine, and dodecyl mercaptan.

11. A pharmaceutical composition for treatment of liver cirrhosis in a subject, wherein the pharmaceutical composition comprises a ligand-bound copper cluster; and
a pharmaceutically acceptable excipient;
wherein the ligand-bound copper cluster comprises:
a copper core; and
a ligand, wherein the ligand binds to the copper core, forming the ligand-bound copper cluster.

12. The pharmaceutical composition of claim 11, wherein the copper core has a diameter in the range of 0.5-5 nm.

13. The pharmaceutical composition of claim 11, wherein the copper core has a diameter in the range of 0.5-3 nm.

14. The pharmaceutical composition of claim 11, wherein the ligand is one selected from the group consisting of thymine, thymine-modified hyaluronic acid (TMHA), L-cysteine and its derivatives, D-cysteine and its derivatives, cysteine-containing oligopeptides and their derivatives, and other thiol-containing compounds.

15. The pharmaceutical composition of claim 14, wherein the L-cysteine and its derivatives are selected from the group consisting of L-cysteine, N-isobutyryl-L-cysteine (L-NIBC), and N-acetyl-L-cysteine (L-NAC), and wherein the D-cysteine and its derivatives are selected from the group consisting of D-cysteine, N-isobutyryl-D-cysteine (D-NIBC), and N-acetyl-D-cysteine (D-NAC).

16. The pharmaceutical composition of claim 14, wherein the cysteine-containing oligopeptides and their derivatives are cysteine-containing dipeptides, cysteine-containing tripeptides, or cysteine-containing tetrapeptides.

17. The pharmaceutical composition of claim 16, wherein the cysteine-containing dipeptides are selected from the group consisting of L(D)-cysteine-L(D)-arginine dipeptide (CR), L(D)-arginine-L(D)-cysteine dipeptide (RC), L(D)-histidine-L(D)-cysteine dipeptide (HC), and L(D)-cysteine-L(D)-histidine dipeptide (CH).

18. The pharmaceutical composition of claim 16, wherein the cysteine-containing tripeptides are selected, from the group consisting of glycine-L(D)-cysteine-L(D)-arginine tripeptide (GCR), L(D)-proline-L(D)-cysteine-L(D)-arginine tripeptide (PCR), L(D)-lysine-L(D)-cysteine-L(D)-proline tripeptide (KCP), and L(D)-glutathione (GSH).

19. The pharmaceutical composition of claim 16, wherein the cysteine-containing tetrapeptides are selected from the group consisting of glycine-L(D)-serine-L(D)-cysteine-L(D)-arginine tetrapeptide (GSCR), and glycine-L(D)-cysteine-L(D)-serine-L(D)-arginine tetrapeptide (GCSR).

20. The pharmaceutical composition of claim 14, wherein the other thiol-containing compounds are selected from the group consisting of 1-[(2S)-2-methyl-3-thiol-1-oxopropyl]-L(D)-proline, thioglycollic acid, mercaptoethanol, thiophenol, D-3-trolovol, N-(2-mercaptopropionyl)-glycine, and dodecyl mercaptan.
